(19) United States
(12) Patent Application Publication
(10) Pub. No.: US 2024/0415429 A1
(43) Pub. Date: Dec. 19, 2024

(54) REAL-TIME INGESTIBLE SENSING CAPSULE

(71) Applicant: UNIVERSITY OF MARYLAND, COLLEGE PARK, College Park, MD (US)

(72) Inventors: Michael STRAKER, Severn, MD (US); Joshua LEVY, Lanham, MD (US); Justin STINE, La Plata, MD (US); Reza GHODSSI, Potomac, MD (US); Luke A. BEARDSLEE, Atlanta, GA (US); Jinjing HAN, Greenbelt, MD (US)

(21) Appl. No.: 18/744,048

(22) Filed: Jun. 14, 2024

Related U.S. Application Data
(60) Provisional application No. 63/508,177, filed on Jun. 14, 2023.

(51) Int. Cl. A61B 5/1473; A61B 5/00; A61B 5/145

(52) U.S. Cl. CPC A61B 5/1473; A61B 5/0004; A61B 5/14507; A61B 5/14546; A61B 2562/125

(57) ABSTRACT

The present disclosure describes various aspects of devices, and methods of making and using the devices, for in vivo sensing of luminal concentration of a compound of interest within a gastrointestinal (GI) tract of a subject. An ingestible housing has an outer profile to pass through the subject's GI tract. A battery and a communication module are contained inside of the housing. A sensor is disposed on an outer surface of the housing. The sensor comprises one or more electrodes configured to contact luminal contacts of the subject's GI tract while the sensor is in motion traversing through the subject's GI tract. A plurality of electrical signals is controlled, and comprises attributes relating to a property of the compound of interest. A continuous output signal is received from the sensor, indicating an extent to which the compound of interest is present in the luminal contents.

### CROSS REFERENCE TO RELATED APPLICATIONS

The present application is based on, claims priority to, and incorporates herein by reference in its entirety for all purposes, U.S. Provisional Patent Application Ser. No. 63/508,177, filed Jun. 14, 2023.

### STATEMENT OF GOVERNMENT SUPPORT

This invention was made with government support under ECCS1939236 awarded by the National Science Foundation. The government has certain rights in the invention.

### TECHNICAL FIELD

The various methods, systems, devices, and processes described herein relate generally to electrochemical sensing and detection within a patient's body. In specific embodiments, the disclosure herein describes embodiments used in sensing molecules and chemicals within the lumen of a patient's GI tract via an ingestible device.

### BACKGROUND

The contents of human and animal gastrointestinal tracts contain a number of substances, compounds and gases of interest, the sensing of which could be valuable toward various health monitoring and/or diagnostic purposes. However, real-time in-vivo/in-situ direct sensing of these substances, compounds and gases throughout has been limited. Instead, devices have been provided to measure their presence in less direct or accurate ways, such as: ex-vivo by measuring GI excretions or emittances; only localized measurement of a GI tissue; or indirect measurement (e.g., by optical camera detection of signs/symptoms suggestive of the presence of a compound/substance/gas of interest). These approaches have certain disadvantages, such as only being able to measure accumulated presence of a compound/gas without knowing where/to what extent it was generated or accumulated along the length of the GI tract, or focusing only on attributes of the GI lining tissue. It would therefore be desirable to have a device that directly measures certain compounds, substances, or gases within GI tract contents (not necessarily GI lining tissue), while a sensor traverses the length of the GI tract (e.g., with the ability to record time series information about sensor readings throughout the GI tract).

As one example, serotonin (5-HT) is a neurotransmitter produced mostly in the gut, which plays an important role in both nervous system and gastrointestinal (GI) function by facilitating signal propagation in the central nervous system (CNS) and enteric nervous system (ENS), and modulating GI motility. Another role of 5-HT is mediating the inflammatory response in immune cells located below epithelial cells that make up the lining of the gut. Due to the myriad of roles that 5-HT takes on, there is much interest in understanding the impact of 5-HT levels on disease and physiological states within the brain and gut.

Evaluating the dynamic behavior of 5-HT in the GI tract is difficult because specific epithelial cells, known as enterochromaffin cells (ECCs), rapidly release and reuptake 5-HT and its quantification is typically achieved through end point analysis of collected samples. Additionally, microbiota in the GI lumen produce and release 5-HT. Detection of 5-HT has also been achieved through electrochemical means utilizing tethered decorated carbon fiber-based sensors. While the current endpoint analysis methods provide a high degree of sensitivity, they lack the ability to evaluate 5-HT dynamics in remote locations in the GI tract. Current designs of sensors require tethering to large equipment and are not ideal for translation to human in vivo applications. Thus, there is a clear need for devices capable of quantifying 5-HT concentration in real-time to elucidate its role in GI function.

As another example, inflammatory bowel disease (IBD) is characterized by chronic inflammation of intestinal tissues and can be associated with dysbiosis of the gut microbiome. Although altered gut bacterial composition has been linked with gastrointestinal (GI) conditions including colorectal cancer (CRC), ulcerative colitis (UC), irritable bowel syndrome (IBS), and metabolic diseases, the physiologic link between dysbiosis and observed pathology has yet to be discovered. Moreover, many digestive diseases share vague and overlapping symptoms such as abdominal pain, nausea, and diarrhea, posing a challenge in delivering a timely diagnosis. Current techniques to diagnose GI disorders in the GI tract rely on endoscopic techniques (e.g., upper endoscopy and colonoscopy) for visualizing and sampling the lumen. More recently, capsule endoscopy has emerged as a noninvasive alternative to traditional endoscopy, capable of accessing distal regions of the small intestines (e.g., jejunum and ileum) and other elusive areas to detect morphological indicators of GI disorders. Existing GI tract sensing platforms employ a variety of sensing technologies including acoustic and optical imaging, as well as physiological readings (e.g., pH, pressure, temperature); however, these modalities fail to detect specific molecular biomarkers of microscopic disease. To better understand the association between microbial activity, inflammation, and disease pathology, sensors that target molecular analytes of interest and in situ technologies enabling their deployment are required.

Breath testing, analysis of flatus and fecal samples, and endoscopic collection are currently standardized methods for quantifying gut microbial H₂S production indirectly; however, noninvasive, real-time solutions are lacking. Unlike H₂ and CH₄, H₂S has a short circulating half-life due to active detoxification and high chemical reactivity, preventing relevant concentrations of intestinal H₂S from reaching the breath. Moreover, H₂S produced by oral microbes (e.g., halitosis) can interfere with breath measurements, further complicating the assessment of the H₂S producing metabolism. Previous clinical studies have successfully quantified H₂S concentrations in the colon from flatus and fecal analysis (0.2-30 parts per million [ppm]), forming definitive associations between the production of H₂S, microbial composition, and diet. However, these methods lack the temporal and spatial resolution required to accurately represent the current state of the intestines. Notably, they obscure key H₂S information in the small bowel, and as a result, precise H₂S concentration levels are unknown. Fully controllable, miniaturized systems capable of real-time, onsite detection of short-lived bacteria-derived inflammatory markers are needed for improved disease management.

Recent advances in flexible electronics, chemical sensors, and smart packaging have contributed to the emergence of ingestible capsule technologies capable of monitoring gaseous molecules in the gut. In general, ingestible gas sensors must operate in both aerobic and anaerobic environments, withstand moisture and caustic conditions, and minimize interference from other electroactive molecules. Previous embodiments have demonstrated wireless ingestible capsules equipped with metal oxide field effect transistor (FET) sensors capable of monitoring oxygen (O₂), H₂, and CO₂ levels in the GI tract. Studies were performed to assess microbiota fermentation processes in response to dietary intervention and exploit the progressively anaerobic conditions in the small and large intestines to track the capsule's position. Metal-oxide FET sensors detect changes in resistance upon adsorption of the target gas and manipulate temperature to modulate selectivity. However, this method is not selective enough to sense trace gases, such as nitric oxide (NO) and H₂S, which are not easily distinguished from predominant gaseous species in the GI tract (e.g., H₂ and CH₄).

### SUMMARY

The present disclosure provides for various configurations of devices and methods that can provide several advantages over the prior art. For example, some aspects of the disclosure may include an ingestible capsule platform for real-time sensing of a substance, compound, or gas within contents of the GI tract in vivo (e.g., 5-HT and H₂S). Such a platform may leverage PCB electronics that have the ability to control one or more sensors to detect a substance/compound/gas of interest, such as by controlling an electrical characteristic of a sensor to detect and record response of the sensor to the luminal contents of the GI tract, achieving miniaturized sensing applications. Some embodiments may use a novel three-electrode flexible 5-HT sensor to conform to the cylindrical shape of an ingestible capsule, or other tailored electrode configurations to adapt to a particular compound of interest. For example, other configurations may be tailored to gas detection, such as examples that may utilize a planar three-electrode sensor featuring a filter (e.g., Nafion filter), allowing the permeation of gases while keeping out liquids and/or solids (of various sizes).

In some aspects, the present disclosure can provide a device for in vivo sensing of luminal concentration of a compound of interest within a gastrointestinal (GI) tract of a subject. An ingestible housing can have an outer profile to pass through the subject's GI tract. A battery and a communication module can be contained inside of the housing. A sensor can be disposed on an outer surface of the housing. The sensor can include one or more electrodes configured to contact luminal contacts of the subject's GI tract while the sensor is in motion traversing through the subject's GI tract. A microcontroller can be in electrically communication with the one or more electrodes. The microcontroller can include a memory having software instructions store thereon, which can cause the microcontroller to control a plurality of electrical signals to the one or more electrodes. The plurality of electrical signals can include attributes relating to a property of the compound of interest. A continuous output signal can be received from the sensor, indicative of an extent to which the compound of interest is present in the luminal contents and contacting the one or more electrodes as the sensor travers the subject's GI tract. Time series information can be transmitting regarding the presence of the compound of interest along the subject's GI tract via the communication module.

In some aspects, the present disclosure can provide a method a manufacturing an in vivo sensor. A capsule can be provided. The capsule can include an ingestible housing, wherein the ingestible housing can be configured to traverse through a subject's GI tract. A battery can be disposed within the capsule. One or more electrodes can be formed on an outer surface of the capsule. The one or more electrodes can be configured to detect luminal concentration of a compound of interest within a gastrointestinal (GI) tract of a subject while the in vivo sensor traverses the GI tract. A microcontroller can be integrated within the capsule and can electrically connect the microcontroller with the battery, the communication link, and the one or more electrodes.

These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1 is a schematic overview of a capsule being ingested by a patient and traveling to the intestines where 5-HT released from both microbiota and gut cells can be sensed according to aspects of certain example embodiments of the present disclosure.

FIG. 2 is an overview of the fabrication of a sensor according to aspects of certain example embodiments of the present disclosure.

FIG. 3A is a rendering of the ingestible capsule system components according to aspects of certain example embodiments of the present disclosure.

FIG. 3B is an image of the pre-assembled system depicting the connections between the internal components according to aspects of certain example embodiments of the present disclosure.

FIG. 4A is a schematic of an experimental setup which a thin film sensor is submerged in a solution and data is recorded via a benchtop potentiostat according to aspects of certain example embodiments of the present disclosure.

FIG. 4B is an example CV response of the sensor of FIG. 4A according to aspects of certain example embodiments of the present disclosure.

FIG. 5A is a schematic of an experimental setup which a capsule is submerged in a solution and data is recorded via an onboard PCB and wirelessly transmitted to a device according to aspects of certain example embodiments of the present disclosure.

FIG. 5B is an example CV response from the capsule of FIG. 5A according to aspects of certain example embodiments of the present disclosure.

FIG. 6A is a schematic representation of a wireless gas-sensing capsule according to aspects of certain example embodiments of the present disclosure.

FIG. 6B is an exploded view of a rendering of the wireless gas-sensing capsule of FIG. 6A according to aspects of certain example embodiments of the present disclosure.

FIGS. 7A-7E illustrate the design and evaluation of an H₂S sensor according to aspects of certain example embodiments of the present disclosure.

FIGS. 8A-8F illustrate graphs of the benchtop characterization of an H₂S sensor according to aspects of certain example embodiments of the present disclosure.

FIGS. 9A-9D illustrate the packaging and characterization of capsule electronics according to aspects of certain example embodiments of the present disclosure.

FIGS. 10A-10E illustrate the electrochemical characterization of an ingestible capsule prototype according to aspects of certain example embodiments of the present disclosure.

FIG. 11 is a schematic diagram of components of a sensing device according to certain embodiments hereof.

### DETAILED DESCRIPTION

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the aspects and embodiments of this disclosure and does not pose a limitation on the scope hereof unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Ingestible capsules have the unique advantage of remotely accessing regions of the gastrointestinal (GI) tract that traditional medical devices are incapable of surveying. For example, ingestible sensors can pass through the GI tract, and thus have the advantage of being able to acquire measurements at multiple locations. Ingestible capsules can be designed to sense the presence of, as well as measurements of, specific liquid, solids, and gases within the lumen of a subject's GI tract. In particular, the design of the ingestible capsule can take into account concentrations of chemicals within the GI tract to accurately survey and visualize microbiota of the lumen. As described above, existing technologies have failed to make full use of the potential advantages of ingestible capsules for certain categories of remote and wireless sensing.

As described herein, there are multiple embodiments for sensing compounds and microbiota of luminal fluid contents, as well as the presence of gases within the GI tract. It should be appreciated by those skilled in the art that the examples and embodiments described herein are for illustrative purposes and that various modifications or changes in light thereof will be suggested to persons skilled in the art and are to be included within the spirit and purview of this application and the scope of the appended claims. In addition, any elements or limitations of any embodiment disclosed herein can be combined with any and/or all other elements or limitations (individually or in any combination) or any other embodiment disclosed herein, and all such combinations are contemplated with the scope of the disclosure without limitation thereto.

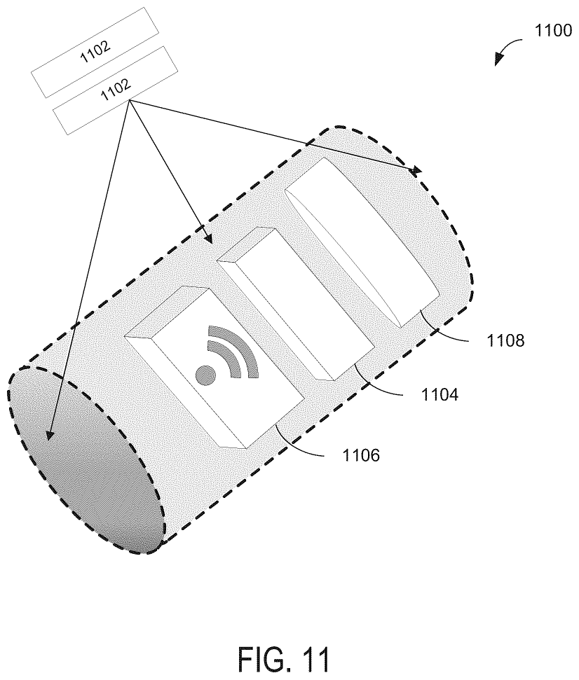

Referring first to FIG. 11, a general schematic diagram of device 1100 is shown, which illustrates attributes and components common to several embodiments. In some examples, the profile of device 1100 may be cylindrical. For example, device 1100 may have a capsule with a diameter between 4-20 mm and length between 1-35 mm. In general, device 110 may have a diameter that is limited by the size of a subject's esophagus, which is the narrowest part of the GI tract. Thus, the size and profile of device 110 may be designed as to allow for comfortable swallowing by a subject. However, in some embodiments, larger sizes may be useful (while still permitting passage through the esophagus), even though swallowing may not be comfortable, as ingestible devices (such as device 1100) may not be swallowed as frequently as other items.

Moreover, the profile of device 1100 may have a smooth surface and/or rounded edges/ends to aid in swallowing the device 1100 and its continuous passage and traverse of the GI tract. In particular, the device 1100 may be a sealed device, without any anchors, arms, extensions, attachments, angles, or profiles that could cause the device 1100 to affix or lodge itself at any given part of the GI tract or otherwise prevent device 1100 from continuously moving throughout the GI tract. In some examples, the device 1100 may be covered or surrounded by a smooth coating or layer. Further, the entire surface of device 1100 may be non-dissolvable, so the profile of the device 1100 remains constant while traversing a subject's GI tract. In other examples, part or all of the surface of device 110 may contain a dissolvable membrane configured to dissolve once reaching a specific portion of a GI tract (e.g., based on PH value of the GI tract contents). For example, the device 1100 may include one or more sensors 1102 that may be coated with a dissolvable membrane, so that the sensors become exposed and begin measurements at an approximate location or region of the GI tract.

Furthermore, the one or more sensors 1102 may be positioned on any portion of the outside surface of device 1100. For example, the one or more sensors 1102 may be disposed on a leading end, a trailing end, a peripheral length of the device 1100, or a combination thereof. In some examples, the one or more sensors 1102 can be made of a flexible and/or thin material. For example, the material of the one or more sensors 1102 may contour to the outside surface of device 1100 to continuously detect and sense. Moreover, the sensors 1102 can include one or more electrodes. In some examples, the one or more sensors and/or one or more electrodes may be configured to sense multiple chemicals or materials within the GI tract at once. In some examples, the one or more sensors may vary in shape an/or size. For example, the one or more sensors may take on a concentric or square shape that can feature inset repetitions to increase a trace length. In some examples, the size of the one or more sensors can correspond to a percentage of the surface area of the device 1100. For example, in instances where a low concentration of a compound or gas of interest is expected, varying the surface area and/or length of a trace may correspond to an increased ability to detect the presence of the compound or gas. In other instances, multiple parallel or redundant sensors may be used to simultaneously measure presence of the same compound or gas. And, the coordination of these sensors may be dynamically varied to adjust gain and/or sensitivity of their readings.

For example, when concentration is intermittent or expected to be localized, the electrodes may operate as multiple independent sensors to increase spatial resolution and likelihood of detection. Similarly, if high noise is expected, having electrodes operate as parallel sensors may also allow for differential analysis to improve detection and noise resolution. Or, if concentration may have a potentially wide range of values, the sensors may operate as one large, connected electrode set, having the ability to record a greater range of values and/or better sensitivity. The inventors have determined that tailoring surface area/trace length of the electrodes is a useful way to tailor a device to achieve better output for a given compound or gas of interest.

As illustrated in FIG. 11, the device 1100 may include a microcontroller 1104. The microcontroller 1100 is in electrical communication with a power source 1108 (to receive operating power); with one or more sensors 1102 (to output and record electrical signals); and a communication link 1106 (to wirelessly communicate data regarding readings from the sensors 1102). In some non-limiting examples, the microcontroller 1104 can be a general-purpose microcontroller, a low power microcontroller, an FPGA, a digital signal processing (DSP) chip, an application specific integrated circuit (ASIC), etc. In some examples, the microcontroller 1104 can include one or more output leads that can allow the microcontroller 1104 to electrically connect to one or more sensors 1102. Thus, the microcontroller 1104 can be programmed to cause electrical signals to be sent to the one or more sensors 1102. For example, the electrical signals can comprise dynamic, variable, and/or constant voltages to the one or more sensors 1102. In some embodiments, the microcontroller 1104 can include one or more input leads configured to receive, read, and measure an electrical signal from one or more electrodes corresponding to the one or more sensors 1102. In some examples, the microcontroller 1102 can also be connected to additional circuit elements. For example, the microcontroller 1104 can be associated with gain amplifiers to generate signals, operational components to filter or scale signals, and/or an A/D converter to acquire signals.

In further embodiments, the microcontroller 1104 may comprise on-board memory (or be connected to a memory within the housing of the device 1100) that stores software instructions thereon. The software instructions may be executed by the microcontroller 1104, to perform tasks associated with outputting various electrical signals to the sensors 1102 and/or reading electrical signals received or detected from the sensors 1102. In some embodiments, the software instructions may cause the microcontroller to output a constant, sweeping, or dynamic electrical signal (e.g., outputting a signal that cycles through various voltage levels, or that adjusts voltage in reaction to signals detected by the sensors 1102). In some embodiments, the software may cause the microcontroller to assess electrical characteristics of the electrical signals read from the sensors to determine the extent to which a given compound or gas of interest may be present. The software instructions may also cause the microcontroller to dynamically adjust its sampling protocol (e.g. sampling rate, signal filtering, signal averaging/normalization, etc.) in response to readings from the sensors 1102. In some embodiments, the microcontroller may also have a hardware- or software-based counter to determine an approximate time associated with readings that are acquired. For example, each value sampled from the output of the sensors 1102 may be associated counter value or time stamp by the microcontroller. The time stamp may comprise a time value, or a counter value that accumulated from a given start time (e.g., a counter that increments from known time value, increments from a first detection, increments from a first signal output of the sensors 1102 after a membrane has dissolved etc.). Based on known average speed of traversal of objects through human/animal GI tracts, the time stamps may be used to determine a general location or region of the GI tract in which the compound or gas of interest was detected or not detected.

In further embodiments, the microcontroller may comprise software that causes it to determine a binary response indicating presence or absence of the compound or gas of interest. In other embodiments the microcontroller may comprise software that calculates a quantitative value regarding the amount of compound or gas detected at a given time. In some embodiments, the microcontroller may also comprise software that calculates a quantitative value representing a cumulative total of compound or gas detected in the GI tract up to that point in time. All or any of these determinations may be stored in memory and/or output via a communication connection as described below.

In some non-limiting examples, the device 1100 may include a communication module 1106. As shown in FIG. 11, the communication module 1106 may be arranged inside of device 1100. In some examples, the communication module 1106 may be a communication link. The communication link 1100 can be configured to enable wireless communication, such as Bluetooth, Bluetooth low energy, near field communication (NFC), medical implant communication service (MICS), or other similar communication protocols.

Also shown in FIG. 11 is a power supply 1108 contained within the device 1100. In some examples, the power supply 1108 can be one or more batteries configured to provide power to any of the other components within the device 1100 (e.g., the one or more sensors 1102, the microcontroller 1104, etc.).

The device 1100 as shown is not meant to be limiting or exclusive of other features described herein below. The profiles, end shapes, membranes, etc. described in any of the fluid sensing or gas sensing examples below could be used generally in any embodiment following the general design of device 1100. For example, the positioning of the sensor in the gas sensing embodiments, below, on a leading end of the capsule (and within a concentrating/concave end profile) could also be utilized for a fluid sensing embodiment.

In some embodiments, the output readings of the electrodes by the sensor do not measure the presence of a compound within a lining of the subject's GI tract. For example, the sensor may only measure the presence or amount of a compound that is freely moving and/or has been released outside of a subject's intestinal tissue. In instances in which the sensor is continuously moving through a GI tract, the sensor's positioning and configuration, as well as the sampling and processing of the sensor signals (as further described below), allow for any contribution to sensor output from a GI tract tissue or lining to be negligible.

### Fluid Sensing

In accordance with one aspect of the present disclosure, an ingestible capsule platform is demonstrated for real-time sensing of certain compounds within the luminal contents (e.g., non-gas, fluid or solid compounds) in vivo. These designs are able to realize the first ingestible capsule capable of investigating neurotransmitter dynamics via electrochemical sensing, overcoming the limitations of traditional sensing approaches.

Below, specific examples and prototypes are described, which may be used in providing a device that can detect and sense specific chemicals and other contents of luminal fluid. It should be understood that the systems and methods described herein that one of skill in the art can use the examples described herein to detect other chemicals and elements of luminal fluid using a portion or all of the components described below. Furthermore, the method of sensing (e.g., potentiometric, amperometric, or conductometric) can also be coordinated with electrode configuration to increase contribution of luminal contents and decrease contribution of luminal tissue. For example, electrodes with a larger surface area that pass through a GI tract continuously are more likely to average a signal.

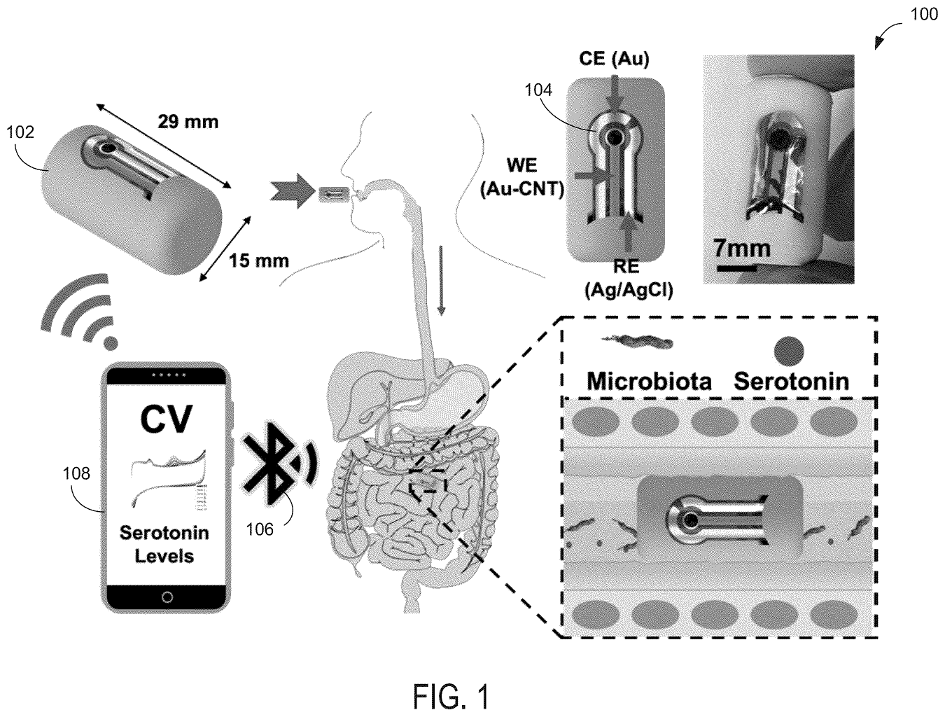

Referring now to FIG. 1, a schematic overview is shown to generally illustrate a system 100 that can include a capsule 102 that is ingested by a patient and traveling through the intestines. The system 100 leverages PCB electronics that can modulate voltage and record current response for miniaturized sensing applications. A novel three-electrode flexible 5-HT sensor 104 can be included that been adapted from an in vitro 5-HT sensing platform to conform to the cylindrical shape of an ingestible capsule. As noted above, the electrodes could also be configured in their shape, size, and material composition to detect other compounds (besides 5-HT). For example, the shape, size, and material composition of the electrode may correspond to with an electroactive compound of interest to vary its electrical performance (electrical sensitivity, linear range, etc.). In some non-limiting examples, increasing the size of the electrode may increase a sensing range or sensitivity of the electrode due to the increase of surface area available for a chemical or compound to absorb into. In some examples, the system 100 can sense serotonin produced by both microbiota and gut cells using sensor 104. The sensor 104 can quantify the serotonin by a gold-carbon nanotube electrode fixed to a shell of capsule 102. In some examples, measurements can be acquired via cyclic voltammetry and transmitted via a wireless communication, such as via Bluetooth 106 to a cellphone 108.

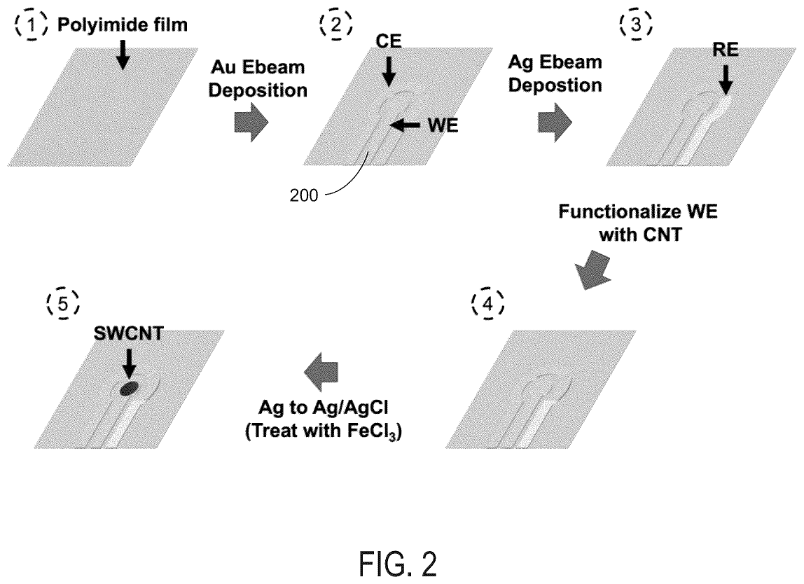

In one aspect, the sensor 104 can be a flexible sensor. In one non-limiting example, the sensor 104 can be fabricated on a 1 mil flexible Kapton® polyimide substrate via a multi-stage metal deposition process. First a shadow mask can be laser cut from paper utilizing a Glowforge Pro laser cutter. Polyimide can be adhered to a silicon wafer via double sided tape and cleaned with acetone, methanol, isopropanol, and deionized water to remove contamination from the surface of the film. Laser cut paper masks can then be taped on top of the film and treated with O2 plasma at 200 W for 60 s to promote adhesion. To construct a working electrode (WE) and counter electrode (CE), 100 nm of gold can then be deposited on the film via electron beam evaporation deposition using the Angstrom Ebeam. A 20 nm layer of chromium can be deposited as a seed layer to promote substrate adhesion. This process can be repeated using a shadow mask laser cut in the pattern of the reference electrode (RE) and depositing 250 nm of silver with a 20 nm titanium seed layer on top of gold. The deposited silver may be chemically treated with ferric chloride (FeCl3) to create a silver-silver chloride reference electrode, such as the Au-CNT sensor 200 as illustrated in FIG. 2. Next, the working electrode can be modified by drop-casting a solution of single walled carbon nanotubes (SWCNT) dissolved in 1:1 Ethanol:N-methyl-2-pyrrolidone (NMP). The modifications may serve to increase the surface area and 5-HT binding sites, in turn increasing sensitivity.

In accordance with one aspect of the present disclosure, a capsule enclosure can be modeled and designed, then printed via fused filament fabrication using acrylonitrile styrene acrylate (ASA) filament. In some examples, the capsule enclosure can be designed with a 15 mm diameter and 1 mm wall thickness. The cylindrical body with sensor port, and two sealing caps can be printed separately to allow for more facile assembly.

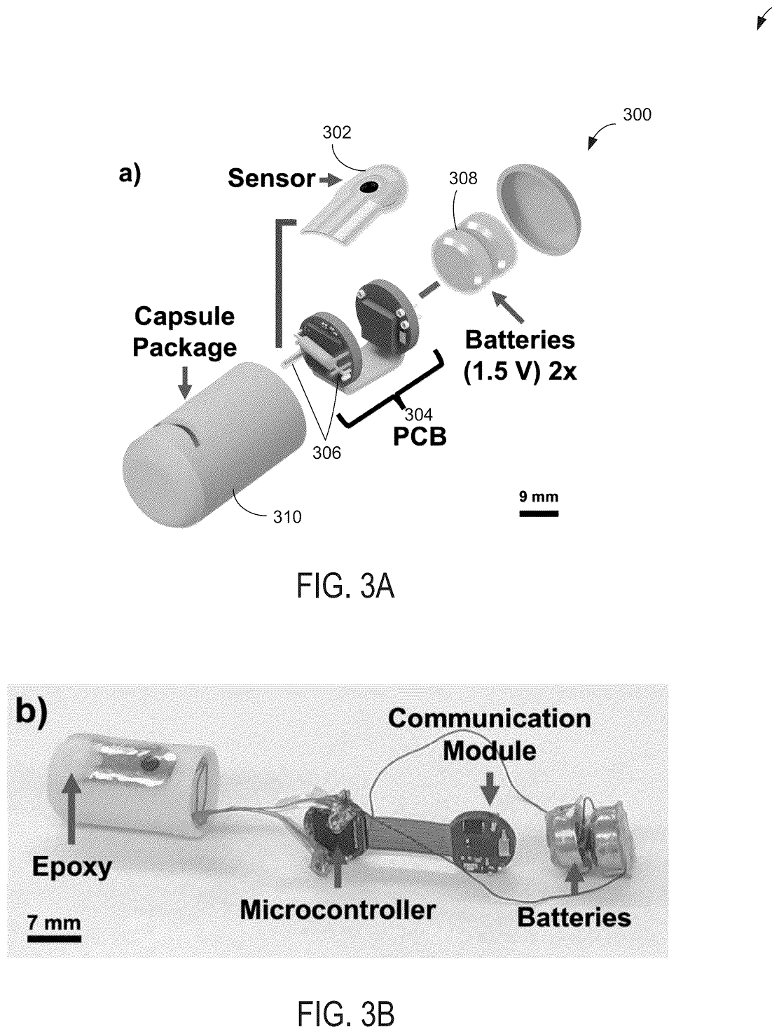

Referring now to FIGS. 3A and 3B, a non-limiting example of an assembly of a capsule 300 is illustrated. In some examples, the assembly of capsule 300 can be achieved by first connecting a sensor 302 to a PCB 304. Wires can be stripped to expose the leads and adhered to the counter, working and reference electrodes of the flexible sensor with copper tape. The adhered leads are pressed to ensure proper adhesion. The wires can then be connected to input pins 306 of the PCB 304 via pin receptacles insulated with polyimide tape. The sensor 302 can then be fed through the slot in the package of capsule 300 and may be adhered to the surface of capsule 300 an adhesive, such as Loctite M21-HP biocompatible epoxy. In some embodiments, capsule 300 can include a power source 308. For example, the power source 308 can include two 1.5 V SR44 W batteries connected in series. In some examples, the power source 308 can then be connected to the PCB 304. The internal components can be packaged into a housing 310. In some embodiments, the housing 310 is a 3D printed housing that can be sealed with the biocompatible epoxy. In some non-limiting examples, experimental data can be relayed via Bluetooth to an iOS device where it is stored. The electrochemical sensor 302 in the system can use cyclic voltammetry (CV) with a voltage sweep between-0.1 to 0.6 V and a scan speed of 100 mV/sec to quantify 5-HT adsorbed to the surface of the working electrode of the sensor, indicating 5-HT concentration in the solution.

However, it is to be understood that the specific processing circuitry components to be used in various embodiments need not be limited to those described herein. In particular, a variety of processors and microcontrollers may be utilized, so long as they meet certain design criteria: they should have a form factor that fits within a given capsule/housing (the size of which can vary, as described below), should have a power consumption that can be addressed by the size and capacity of batter that can be included in the housing, and should be able to have sufficient processing speed to acquire and transmit data from the sensor. In yet further embodiments, the microcontroller may have a memory associated with it that permits more complex routines to be run on the processing unit of the microcontroller. For example, the microcontroller may selectively change or sweep frequencies of the interrogation current (whether autonomously or via pre-set programming), may respond to control instructions from an external device, and may react to the types of signals it determines.

Similarly, rather than a mere antenna to act as a transmitter, a transceiver and/or separate receiver may be utilized. These embodiments would allow for bi-directional communication between an external control device and the capsule device.

### Example Serotonin Sensing Capsule Set-Up and Experimentation

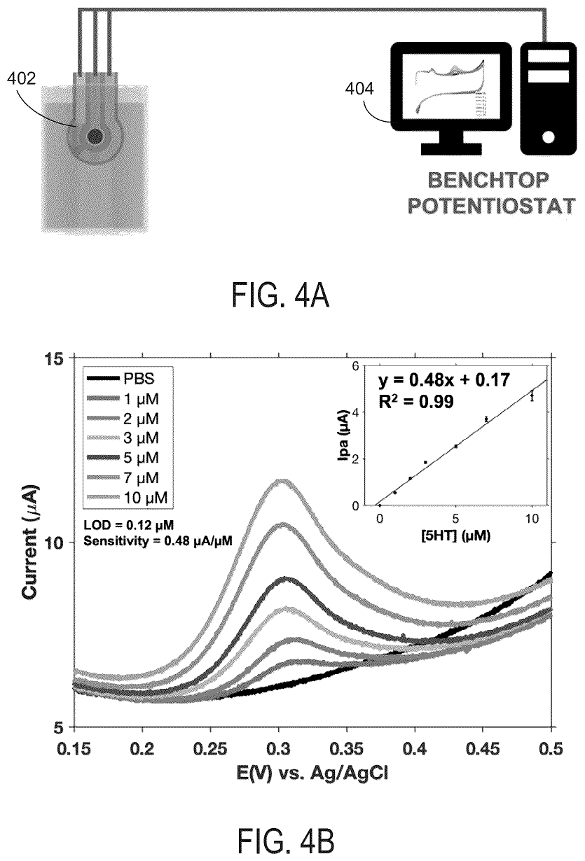

In a non-limiting example, an isolated flexible 402 sensor can be characterized in solutions of 5-HT dissolved in 1× phosphate buffer saline (PBS), as illustrated in FIG. 4A. The sensor 402 may be submerged in the 5-HT solution. Next, a benchtop potentiostat 404 can be used to record CV response in solutions of 5-HT concentrations of 0, 1, 2, 3, 5, 7, and 10 μM, as seen in the non-limiting example results illustrated by FIG. 4B. In some examples, the sensor 402 may perform a voltage sweep between-0.1 to 0.6 V and a scan speed of 100 mV/sec with a 1 min accumulation time.

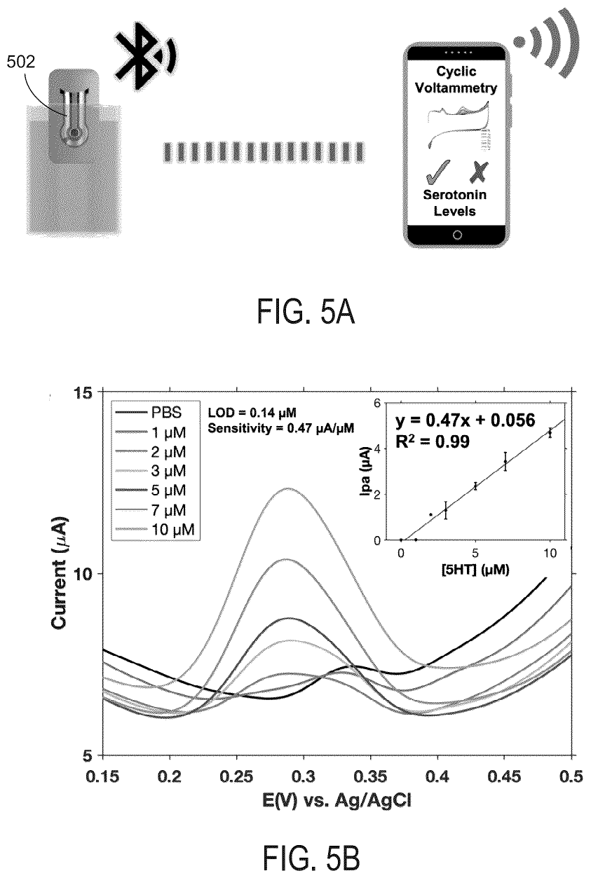

In a non-limiting example, CV can be conducted using a sensor 502 with a wireless PCB within the same 5-HT concentration range to evaluate the performance of the combined PCB-sensor system, as shown in FIG. 5A. Similar to the benchtop potentiostat, the electrochemical sensor in the system may use cyclic voltammetry (CV) with a voltage sweep between-0.1 to 0.6 V and a scan speed of 100 mV/sec to quantify the concentration of 5-HT adsorbed to the surface of the working electrode (WE) of the sensor 502. In one example, the accumulation time was set at 1 min to match isolated sensor protocol. Data was post processed via low pass filtering to reduce noise and analyzed.

Next, CV was performed in a solution containing 5-HT concentrations from 1-10 μM via a benchtop potentiostat to evaluate the performance of the isolated flexible sensor 502. The results show a linear relationship between the anodic current peak (Ipa) at ˜0.3 V, with a sensitivity of 0.48 μA/μM and a limit of detection (LOD) of 0.12 μM. A linear fit of the data showed an R2 value of 0.99.

The CV response utilizing the wireless PCB was comparable to that of the benchtop measurements, with a sensitivity of 0.47 μA/μM and a LOD of 0.14 μM. A shift in peak potential is observed from the 0.3 mV to 0.28 mV when compared to CV response measured with the benchtop potentiostat. The dynamic range of the device was from 2 μM to 10 μM. The linear correlation of the Ipa response confirms that the sensor was operating in its linear range.

### Gas Sensing

In accordance with another aspect of the present disclosure, an ingestible capsule platform is demonstrated for real-time sensing of certain gases within the luminal contents in vivo. These designs are able to realize the first ingestible capsule capable of investigating gas production within a subject's GI tract (intra-luminal gas measurements, rather than measuring gut gas production ex-vivo) via electrochemical sensing, overcoming the limitations of traditional sensing approaches.

Below, specific examples and prototypes are described, which may be used in providing a device that can detect and sense specific gases which are present within GI luminal contents. It should be understood that the systems and methods described herein that one of skill in the art can use the examples described herein to detect other gases elements of luminal fluid using a portion or all of the components described below.

Hydrogen sulfide (H₂S) is a gaseous inflammatory mediator and important signaling molecule for maintaining gastrointestinal (GI) homeostasis. Excess intraluminal H₂S in the GI tract has been implicated in inflammatory bowel disease and neurodegenerative disorders; however, the role of H₂S in disease pathogenesis and progression is unclear. The capsule described herein can also be capable of performing electrochemical gas-sensing and can enable real-time, wireless amperometric measurement of H₂S in GI conditions.

In accordance with one aspect of the present disclosure, a gold (Au) three-electrode sensor can be modified with a Nafion solid-polymer electrolyte (Nafion-Au) to enhance selectivity toward H₂S in humid environments. For example, the Nafion-Au sensor-integrated capsule may illustrate a linear current response in H₂S concentration ranging from 0.21 to 4.5 ppm (R2=0.954) with a normalized sensitivity of 12.4% ppm-1 when evaluated in a benchtop setting. In some examples, embodiments of sensors described herein can prove to be highly selective toward H₂S in the presence of known interferent gases, such as hydrogen (H₂), with a selectivity ratio of H₂S:H₂=1340, as well as toward methane (CH₄) and carbon dioxide (CO₂). In some examples, the packaged capsule can further demonstrate reliable wireless communication through abdominal tissue analogues, comparable to GI dielectric properties. In further examples, assessments of sensor drift and threshold-based notification can be performed for in vivo application.

Previous electrochemical detection of H₂S was often incompatible with the intraluminal environment, and lack the selectivity, sensitivity, and low-voltage operation essential for integration with ingestible capsule platforms. Electrode materials like platinum and modified carbon that are sensitive to H₂S due to their affinity to H₂, do not provide the necessary selectivity for intestinal H₂S sensing, as O₂, nitrogen (N₂), CH₄, CO₂, and H₂ (up to 50%) make up 99% of intestinal gas. Au is sensitive to H₂S via a high adsorption affinity to sulfur molecules, and is not reactive to H₂. Mubeen et al. utilized this sensing mechanism to develop an H₂S sensor employing single-walled carbon nanotubes (SWNT) decorated with Au nanoparticles. However, the sensor required a bias voltage ranging from 1-4 V for the desorption of sulfur(S) from the Au electrode, resulting in a slow response and recovery time. When Au is exposed to H₂S, sulfur atoms covalently bond with the Au surface. Au—S covalent bonds significantly reduce the work function of Au and require a substantial amount of energy to reverse. Therefore, while Au alone can be used to sense H₂S, further modification of the working electrode is can be an important step in achieving the sensitivity and recovery characteristics to sense trace concentrations of intraluminal H₂S gas in real-time.

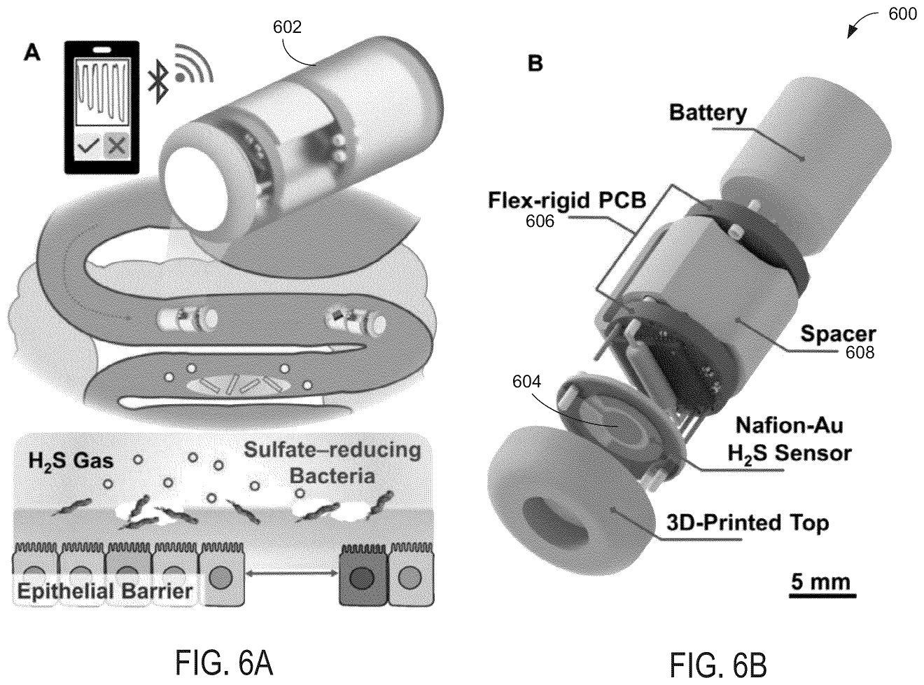

Referring to FIGS. 6A and 6B, a non-limiting example of a system 600 including a fully integrated gas-sensing ingestible capsule 602 for real-time, wireless detection of H₂S in the GI tract is illustrated. The system 600, as shown in FIG. 6B, features a thin, flexible electrochemical sensor 604. In some examples, the sensor 604 can utilize a Nafion solid-state polymer electrolyte (SPE) and gold (Au) sensing electrode (Nafion-Au) to selectively monitor physiological H₂S concentrations. The capsule 602 can include electronics that integrate an electrochemical analog front-end (AFE) to facilitate amperometric measurements and a Bluetooth Low Energy microcontroller (BLE-MCU) for data processing and wireless transmission. In some examples, various components can be mounted on a flex-rigid printed circuit board (PCB) 606 and encapsulated in a polymer, such as a soft polydimethylsiloxane (PDMS) polymer. A 3D-prited spacer unit 608 can be placed between the PCB 606 to allow for integration of future system modalities. In some non-limiting examples, the resulting overall capsule can be a form factor of 14×34 mm². The size and shape may be compatible with large porcine animal models and can be readily scaled for implementation in vivo.

### Example Hydrogen Sulfide Capsule Setup and Experimentation

In one non-limiting example, pretreatment of the Nafion SPE to increase the conductivity and hydration capacity of the membrane was investigated. The operability of the ingestible capsule prototype was evaluated in a custom humidity-controlled gas testing chamber. Electrochemical characterization demonstrated linear detection of H₂S over concentrations ranging from 0.21 to 4.5 ppm (14.6 μm) and selectivity to H₂S in the presence of 100-fold greater concentrations of interfering gases, namely H₂, carbon dioxide (CO₂), and CH₄. Some embodiments of the device address system integration challenges associated with amperometric detection of H₂S in the GI tract and proves capable of identifying fluctuations of H₂S levels under simulated GI environment conditions.

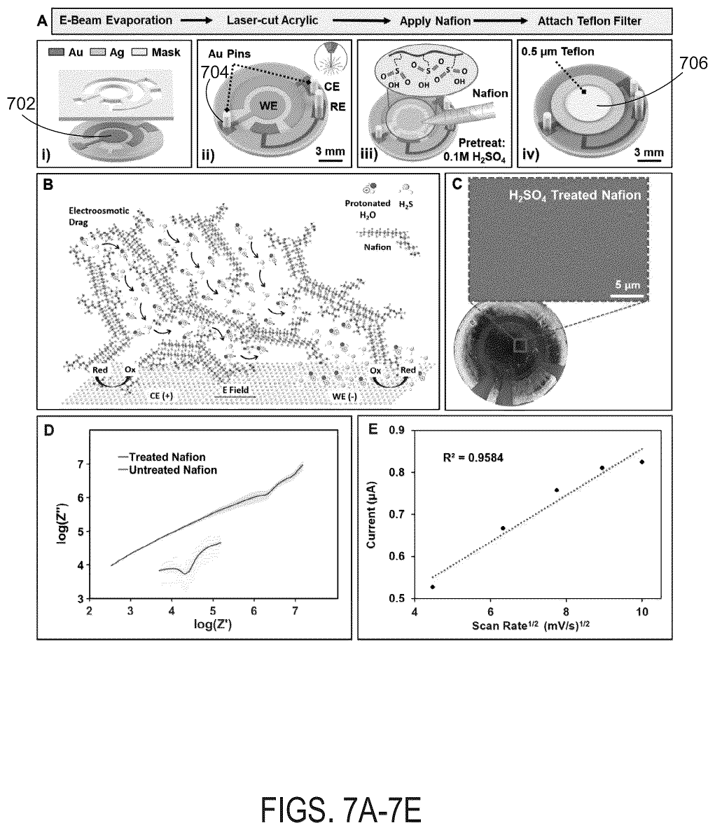

An electrochemical H₂S sensor 702 (Ø=12 mm) in one experiment comprised of a thin-film Au working electrode (WE, Ø=4 mm), Au counter electrode (CE), and a silver (Ag) reference electrode (RE) evaporated on a flexible polyimide substrate (Kapton, 1 mil), as illustrated in box i of FIG. 7A. Laser-cut acrylic wells were then attached to the sensor 702 to serve as a reservoir for forming the Nafion membrane from dispersion. To integrate the sensor 702 with capsule electronics, Au pins 704 were connected to each electrode contact pad using Ag epoxy, as shown in box ii of FIG. 7A. Then, 5% w v-1 Nafion dispersion (EW, 1100 g eq-1) was drop-cast into the acrylic reservoir to sufficiently cover the entire sensor area (Ø=6 mm), followed by subsequent pretreatment with sulfuric acid (H₂SO₄), as shown in box iii of FIG. 7A. Finally, a Teflon membrane 706 (Ø=6 mm, pore size: 5 μm) was lightly pressed into contact with the treated Nafion film and sealed, as shown in box iv of FIG. 7A. The Teflon membrane 706 can function as a gas-permeable, liquid-impermeable interface between the Nafion and the external environment, limiting sensor corrosion and fouling. In other embodiments, the membrane 706 may be semi-permeable to gas or semi-permeable to liquid (e.g., permitting passage of only a certain size of molecule).

Nafion is a chemical-resistant perfluorinated cationic exchange polymer (CEP) with exceptional membrane-forming capabilities and is often used as an SPE to enhance the sensitivity, conductivity, and recovery of the fabricated sensor. Dispersion and baking of Nafion crosslinks the polytetrafluoroethylene (PTFE) backbone to form stable clusters of hydrophilic sulfonic acid end groups surrounded by hydrophobic PTFE. The hydrophilic regions expand as the membrane is hydrated and connect into a network of ionic conduction channels if sufficiently hydrated. Water uptake, ion exchange capacity, and ion conductivity together determine the conductivity of the Nafion membrane. While this hydration dependence is problematic for some applications, the high-water content of the GI tract may provide sufficient humidity levels to hydrate the Nafion and ensure conductivity throughout capsule transit. When a hydrated CEP is biased, protonated water molecules (e.g., H5O2+, H9O4+) flow from the anode to the cathode of the sensor, simultaneously transporting H₂S and other small gaseous molecules in the process, as illustrated in FIG. 7B. This electroosmotic drag results in an accumulation of H₂S molecules and protons at the surface of the WE, reducing the work function of Au and increasing the reduction current.

The conductivity of the Nafion membrane increases with decreasing thickness until reaching the thin-film regime (<10 μm). To achieve a uniform Nafion membrane at the desired thickness, removal of the solvent and surface interactions between the Nafion dispersion and Au substrate can be important. Slowly evaporating the Nafion dispersion allows for an even distribution of hydrophilic and hydrophobic regions in the Nafion, resulting in higher conductivity. Therefore, the rate of solvent evaporation was carefully controlled in order to minimize the risk of cracks forming in the film. To accomplish this, an additional solvent (80% ethanol) was introduced into the sealed container and allowed it to evaporate for 24 h. The Nafion-Au sensors were then baked in a furnace at 80° C. for 1 h to remove any remaining solvent subsequently placed in a sealed humid container for 24 h to rehydrate.

To identify a suitable pretreatment procedure for protonation of the Nafion coated Au electrodes, varied concentrations of H2SO4, treatment time intervals, and solution temperatures were investigated. Preliminary amperometric measurements were recorded for each combination of H2SO4. While sensors treated with 1.0 m H2SO4 exhibited higher linearity and a consistent bias voltage, they displayed low conductivity and poor sensor durability regardless of test conditions. This outcome was likely due to a combination of insufficient acid protonation time and corrosion of the Cr seed layer beneath the AuWE and Ag RE, leading to reduced electroactive surface area. When testing various protonation times for 0.1 m H2SO4, a protonation time of 24 h or less resulted in low membrane conductivity. In contrast, a 48 h protonation time produced stable, conductive sensors without corrosion of the Cr seed layer. Therefore, the Au-Nafion sensors were treated by dispersing 0.1 m H2SO4 (50 μL) and resealing the sensor in a humid container for 48 h to allow the acid to protonate the film, followed by rinsing with deionized (DI) water. This resulted in an evenly dispersed Nafion membrane that demonstrated consistent conductivity and mechanical durability once baked and treated, providing a final film thickness of ˜45 μm. Inspection of the WE via a scanning electron microscope (SEM) confirms a uniform membrane without cracks or deformities in the Nafion or electrode which would impact conductivity and sensor durability, as illustrated in FIG. 7C. Electrical impedance spectroscopy (EIS) was utilized to characterize the Nafion-coated sensors. FIG. 7D shows the average impedance of the Nafion-Au sensor before and after acid treatment, confirming significant improvement in the membrane conductivity, according to one non-limiting example. While the acid pretreatment is not expected to impact shelf life, it is important to store protonated Nafion membranes in environments without fluctuations in humidity. The sensors were stored at room temperature in a dry environment away from direct sunlight until use. As a result, the sensors were able to retain conductivity for over 12 h of testing.

The electrochemical response of the fabricated H₂S gas sensor was evaluated using a benchtop potentiostat and custom gas-testing setup. Specific gas concentrations of various intraluminal gas species were achieved by controllably venting gas into a plastic test chamber (2400 mL)—diluted with air—until the desired concentration was reached. To preserve the conductivity of the Nafion membranes, all experiments were performed under humid conditions. This was achieved by periodically venting water vapor into the chamber for 60 s along with the gaseous analytes. Cyclic voltammetry (CV) was applied in the potential range of +0.2 and −0.2 V (scan rate: 100 mV s-1) to observe cathodic currents. The cyclic voltammogram of the fabricated Nafion-coated H₂S sensors was presented at different gas saturation states: ambient air, 2.3 and 4.5 ppm of H₂S. An increasingly negative reduction current response corresponding with increased H₂S concentration was observed between −0.1 and −0.2 V (vs Ag), indicating a range of suitable bias voltages to detect H₂S. Therefore, subsequent amperometric measurements were performed at a bias voltage of −0.2 V to maximize the sensitivity of the Nafion-Au sensor to H₂S, while avoiding potential interference from reduction peaks of O2. The influence of scan rate on the sensor response to H₂S was also examined by comparing cyclic voltammograms of different scan rates when the sensor was exposed to 2.3 ppm of H₂S, as shown in FIG. 7E. The results demonstrated that the current response corresponded to changes in H₂S gas, and that the system was diffusion controlled (linear relationship between current and the square-root of scan rate).

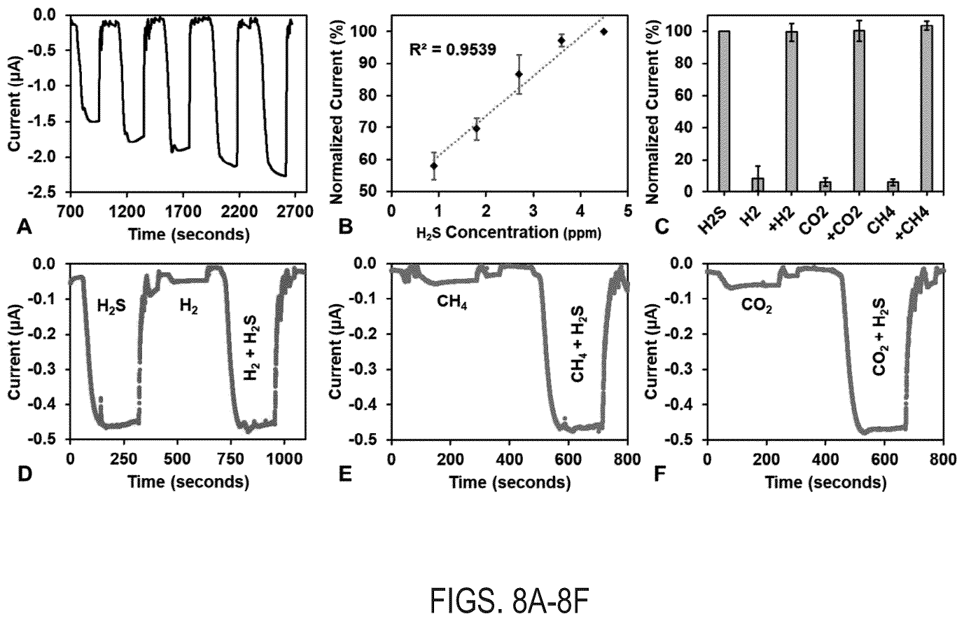

To generate a calibration curve for the H₂S sensor, concentrations ranging from 0.9 to 4.5 ppm of H₂S were introduced into the custom gas chamber at 0.9 ppm intervals following a 10 min warm-up time. Electrochemical sensors typically require a brief warm-up time to allow the electrodes and electrolyte to equilibrate under a voltage bias. Negligible differences in sensor readout due to changes in temperature when operating either at ambient (22° C.) or internal body temperatures (37° C.) were observed, thus all experiments were conducted at room temperature. As the sensor is intended to continuously measure H₂S, the voltage bias will be applied at least 10 min before ingestion. The amperogram of a single sensor is shown in FIG. 8A, where increasing and decreasing sensor current output reflects both an increase and decrease in H₂S concentration. After H₂S was vented into the chamber, the current increased rapidly until reaching saturation after 3 min (including infill time). To return the chamber to an ambient baseline condition (air), the chamber contents were removed using a vacuum pump. The required time to return the sensor to its baseline value was dependent on the gas concentration in the chamber, though purging for three 20 s pulses with a 15 s pause between them was sufficient for all test values and preserved the sensor's lifetime. The resulting calibration curve, shown in FIG. 8B, depicts the normalized average current response from multiple Nafion-Au H₂S sensors (N=3), where 100% represents 4.5 ppm H₂S. A linear current response (correlation coefficient R2=0.954) and sensitivity of 12.4% ppm-1 was observed with slight current saturation starting at 3.5 ppm. At 5.4 ppm, there was no discernable difference between an increase in H₂S and noise. Differences in the current response, shown by FIG. 8B error bars, can be attributed to slight variations in Nafion membrane thickness, hydration, and the density of Nafion clusters in the membrane. As calibration of Nafion-based sensors is highly humidity dependent, the H₂S sensors were calibrated in ˜100% humidity, based on the expected humidity of the GI tract.

The selectivity of the Nafion-Au sensor was characterized between H₂S and potential interferent gases in the GI tract. Amperometric measurements of 4.5 ppm H₂S were performed in the absence and presence of 415 ppm CH₄, 16 650 ppm CO₂, and 500 ppm H₂, respectively. The resulting amperograms demonstrated that accurate detection of H₂S remained mostly unaffected in the presence of greater than 100-fold concentrations of CH₄, CO₂, and H₂, illustrated in FIG. 8C. FIGS. 8D-8F show the sensor response for each gas and indicate less than 10% variation when comparing the combined signal response from H₂S and interferent gases to the H₂S baseline. Overall, the fabricated H₂S sensor demonstrated selectivity to H₂S in the presence of all interferent gases, including H₂, and outperformed the commercial 3SP—H₂S −50 SPEC-H₂S sensor which showed elevated current response to H₂.

### Example Ingestible Gas-Sensing Capsule Design

In one experiment, the inventors developed miniaturized potentiostat electronics (Ø=12 mm) capable of performing amperometric measurements to facilitate in situ sensing using the Nafion-Au H₂S sensor. The system incorporates several commercial off-the-shelf (COTS) components (operated at 3.3 V): 1) an electrochemical AFE, AD5941, to excite the electrochemical sensor and record resulting current values, 2) a BLE-MCU, BGM13S, and external 2.45 GHz ceramic chip antenna, WLA.01, for wireless data acquisition, and 3) a 3.0 V, 160 mA h lithium manganese dioxide (Li—MnO2) coin-cell battery (Ø=11.6), 2L76, and magnetic reed switch to shut off the device when not in use. Inter-device communication is handled by a serial peripheral interface (SPI) for system configuration and to facilitate data transfer between the AFE and BLE-MCU. The BLE-MCU was programmed to receive and transmit data wirelessly via BLE using the EFR Connect phone app and a custom GATT profile. This allowed for remote calibration and initiation of amperometry gas measurements, as well as control of the energy modes of the on-board electronics.

When an amperometric measurement is initiated, the AFE applies a bias voltage across the WE and RE. The resulting current response between the CE and WE are passed through a transimpedance amplifier (TIA), where the signal is converted to a voltage and amplified. Data are sampled every 100 ms and digitized via an internal analog to digital converter (ADC), then stored temporarily to a ferroelectric random-access memory (FRAM) onboard the AFE. Simultaneously, the BLE-MCU periodically interrogates the status of the FRAM (100 ms interval) to determine whether data are available for wireless transmission to the phone. In this active transmission mode, the device consumes an instantaneous current of 10 mA and average of 3.5 mA. Battery life-time calculations estimate a 29 h capsule lifetime under continuous operation of the wireless communication and amperometric gas sensor. While the capsule size and form factor presented here is larger than existing FDA-approved ingestible electronics, the current dimensions are acceptable for in vivo large porcine models. Ingestible capsules of varied size and function, employed in vivo for testing in porcine studies were summarized. The main limiting factors for further scaling the H₂S sensing capsule are the size of the COTS electronic components and compatibility of the battery chemistry. Bluetooth LE has an instantaneous current consumption of ˜10 mA (at +0 dB antenna gain), which prevents the use of alternative safe battery chemistries, such as silver oxide (Ø=9.5 mm), without greatly reducing the wireless transmission rate (10 s). Example Characterization of the Packaged Gas-Sensing Capsule

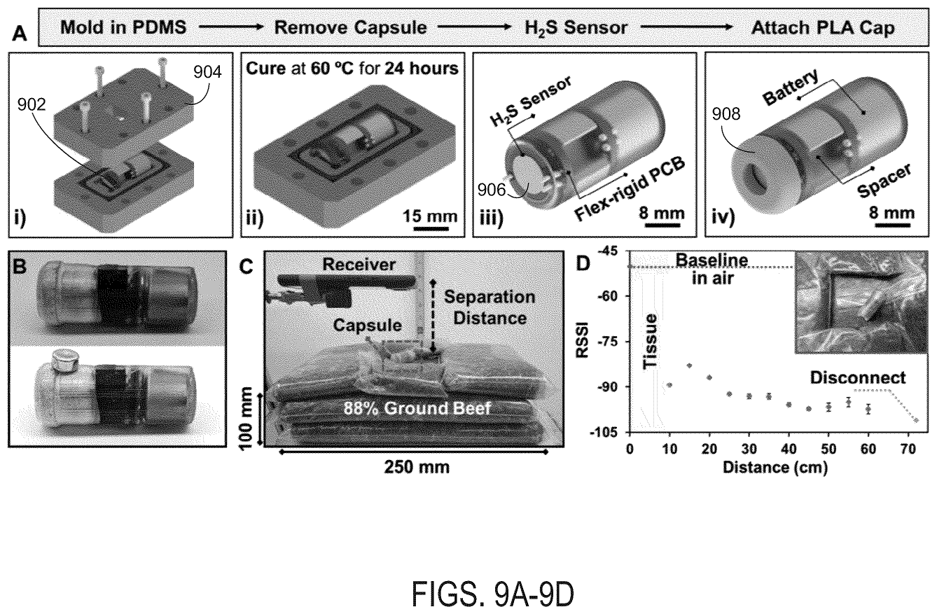

To investigate the characterization of the capsule, in one experiment The benchtop validated sensor was integrated with the capsule electronics and packaged in two stages: 1) encapsulation of the electronic components in a soft polymer and 2) attachment and sealing of the H₂S sensor to preserve the hydration of the Nafion SEP during subsequent evaluation of the packaged device. Here, capsule electronics 902 were encapsulated in PDMS molds 904 at a 10:1 monomer to curing agent ratio and baked at 65° C. for 24 h prior to removal (shown in box i of FIG. 9A). While there are concerns regarding the potential for liquid uptake through PDMS molds 904, modified curing parameters and additional coating of Parylene C have been shown to minimize this effect. The packaged capsule has a resulting 14×34 mm² cylindrical form factor (shown in box ii of FIG. 9A). FIG. 9A-iii illustrates an example modular attachment of a H₂S sensor 906 and molded capsule electronics. Finally, a 3D-printed cap 908 (Ø=14 mm) made from polylactic acid (PLA) was sealed to the PDMS capsule with a biocompatible epoxy to cover the Teflon protected sensor while still allowing gas to diffuse from the external environment (shown in box iv of FIG. 9A). When not in use, the capsule system was placed on an external magnet to disconnect the battery via the magnetic reed switch. This is demonstrated in FIG. 9B, where a small neodymium magnet 910 (1 cm2, ˜600 mT) placed less than 20 mm distance from the magnet reed switch is sufficient to disconnect the system as indicated by an indicator, such as a blue light emitting diode (LED).

### Example Bluetooth Characterization for Ingestible Operation

Wireless medical devices, including ingestible capsules, wearable sensors, and implantable devices typically operate within the 405, 915 MHz, and 2.45 GHz ISM frequency bands. Bluetooth, which operates at 2.45 GHz, is a low-power, point-to-point communication protocol that permits a compact antenna footprint and is widely compatible with consumer electronic products. Known challenges of wireless communication for ingestible electronics include inefficient signal propagation within the GI tract, due to the high radio frequency (RF) signal attenuation in abdominal tissues (∈r=52.8), high current consumption compared to sensing circuitry, and limited high-density battery chemistries that support high current consumption (≈10 mA) in a capsule form factor. To simulate the expected RF attenuation in vivo, the capsule was surrounded by 100 mm of ground meat (88% lean, 12% fat) on all sides, and wireless signal attenuation testing was performed. The dielectric properties of the tissue analogue can be modified to precisely mimic the permittivity of human abdominal tissues using liquid phantoms or ballistic gel. A 100 mm thick layer of ground beef was utilized as a worst-case approximation to account for potential variations in dielectric constant between ground meat (∈′=45-55) and abdominal muscle. This is because most signal attenuation occurs through abdominal muscle, which has an average thickness of only 5-10 mm.

In one experiment, the inventors restricted the antenna gain of the BLEMCU to +0 dB gain in order to evaluate Bluetooth with minimum power consumption, though transmission at +8 dB can be utilized sporadically to extend communication distance. The relative received signal strength (RSSI) between the device and a smartphone (Google Pixel 6, BLE) was recorded at various heights above the capsule. The separation distance from the phone was increased at 50 mm intervals until connection failure (as shown in FIG. 9C). FIG. 9D presents the one example graph illustrating the averaged RSSI values through 100 mm of the tissue analogue. Reliable data transmission was maintained up to 720 mm, with a max RSSI of −99 dBm before disconnecting from the phone (antenna sensitivity is −100 dBm). An average RSSI value (N=30) was recorded at each height, showing an exponential decrease from the average baseline (100 mm separation) of −52 dBm in air taken as the system loss.

Example Electrochemical Measurement with Packaged H₂S Gas-Sensing Capsule

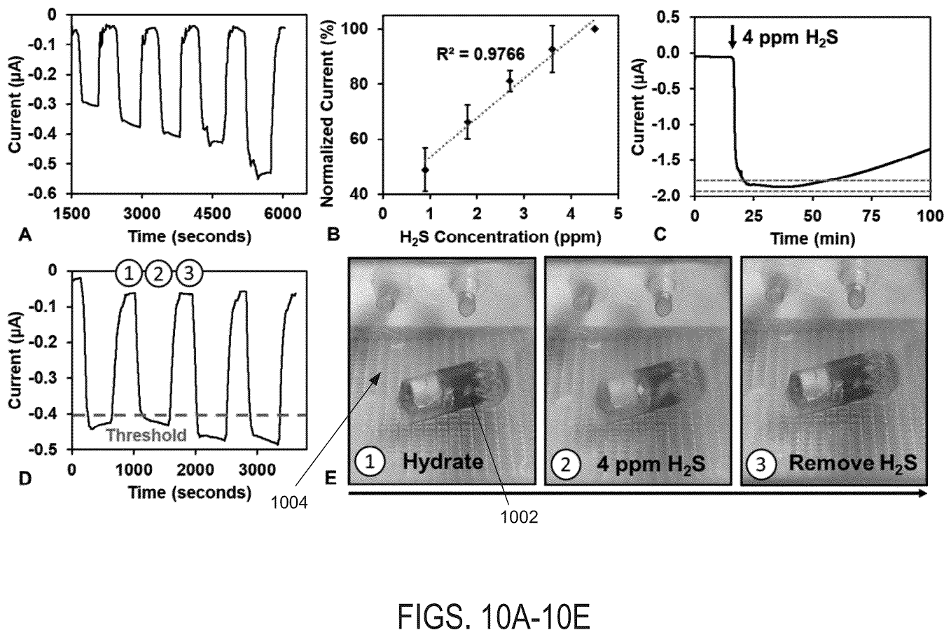

FIG. 10A illustrates a non-limiting example of resulting amperometric measurements for the gas-sensing capsule with a bias voltage of −0.2 V (vs Ag), when tested under the same conditions as the benchtop sensors. A 14.6% ppm-1 current response was observed, confirming that the capsule packaging, wireless communication, and mesoscale electronics does not impact sensor performance. A example of a calibration curve, shown in FIG. 10B, was generated by averaging the normalized current response from Nafion-Au H₂S sensors (N=3) equipped to the gas-sensing capsule, where 100% represents 4.5 ppm H₂S. A linear current response (correlation coefficient R2=0.9766) matched the saturation characteristics of the benchtop sensor characterization and a 0.13 ppm limit of detection (LOD) was calculated, indicating that the packaging did not have a negative impact on sensor response time or saturation characteristics. Benchtop testing confirmed H₂S selectivity and sensitivity with the Au-Nafion sensor. However, in this controlled simulated environment the effects of the accumulation of proteins and lipids on the surface of the Teflon filter were not assessed, which could alter its diffusive properties leading to a deteriorated sensor response. Endoscopy capsules like the Pill-Cam provide an outlook of the anticipated conditions, supporting the assumption that the sensor may be partially submerged during in vivo measurement throughout the GI tract. The attached Teflon filter protects the Nafion membrane from direct exposure to acidic gastric fluids and alkaline bile salts (pH 7-8) in the small intestine, a common concern with ingestible electronics, while also contributing to diffusivity through its pore size and hydrophobicity of the Teflon filter surface. Future efforts will focus on validating the gas-sensing capsule in solutions with acidic and slightly basic pH, as well as in simulated in vitro fluids, such as intestinal digesta or fistulated rumen. This evaluation will be conducted over the course of 12-24 h to evaluate sensor stability and the necessity for recalibration.

To demonstrate the utility of the gas-sensing capsule system to identify and respond to target gas concentrations, in one example, H₂S-driven feedback control was developed to trigger other downstream capsule functions. Previous sensor calibration curves were utilized to determine a suitable threshold concentration level and corresponding current response that represented an elevated H₂S concentration. From the literature, trace H₂S concentrations for patients with UC or SRB-related SIBO may exceed 30 ppm of intraluminal H₂S in the colon, though in healthy patients has been shown to be substantially less (˜0.2 ppm). Therefore, high sensitivity and selectivity for trace H₂S levels in the large and small intestine is essential for correlating differences in healthy and diseased states. In a clinical setting sensor drift must be accounted for when determining a suitable threshold current value in real-time. Amperograms quantifying sensor drift were recorded to evaluate the stability of the Nafion-Au sensor, as illustrated in by the example plot in FIG. 10C. To accomplish this, the inventors placed sensor system 1002 in a test chamber 1004 and the H₂S concentration was fixed at 3.6 ppm for 2 h. The sensor system 1002 exhibited negligible drift for 30 min before the saturated current began to linearly drift back toward the sensor baseline at a rate of 0.1 nA s-1 (R2=0.98), Sensor drift is a known issue with Nafion sensors, and is possibly due to back diffusion of water out of the Nafion membrane. It is hypothesized that while electro-osmotic drag, H+ transport, and water transport contribute to the selective gas-diffusing properties of Nafion, water gradients within the membrane can also cause water to diffuse back out of the Nafion membrane after extended periods of activation. Membrane thickness, acid treatment, and substrate porosity significantly impact the prevalence of back diffusion, and therefore sensor drift.

Considerations for progressively updating the threshold value may be performed, either by introducing a linear correction factor to account for sensor drift exceeding acceptable 5% deviation in current response, or by delaying when the measurement occurs using pH targeting to localize specific sections of the small bowel amenable to the sensor's operational lifetime. In one non-limiting example, threshold triggered signaling was demonstrated by placing the packaged capsule system 1002 into the gas testing chamber 1004 and repeatedly modulating the H₂S concentration between 0 and 3.6 ppm following saturation. FIG. 10D shows an example of the corresponding amperogram, summarizing this implementation of the threshold value. Throughout the experiment the capsule was programmed to wireless alert an app when the current threshold value was surpassed. A video demonstrating when the H₂S concentration level surpassed 3.0 ppm, indicated by a blue LED, was recorded with a phone. The subsequent hydration, H₂S infill, and H₂S purge events are shown in FIG. 10E, highlighting the potential of the system for deployment of complex monitoring and interventions in the GI tract.

To evaluate the H₂S sensing platform in vivo, and ultimately for clinical use, several remaining challenges must be addressed, including system shelf-life, sensor stability, sensor drift, sterilization, and capsule miniaturization. With proper storage Nafion is expected to have a shelf life of up to 2 years, attributed to its chemical and mechanical durability. Future testing following an extended time in storage can be important in determining if the sensors require recalibration before ingestion. As calibration using a gas-testing chamber would be unrealistic in a clinical setting due to sterilizations concerns, special UV treatments could be performed to preserve Nafion membrane conductivity over time, potentially eliminating the need for recalibration. The effects of such treatments and similar sterilization protocols on the semipermeable Teflon membrane filter require further investigation before adaptation of the sensing capsule for clinical use cases. Additionally, the PDMS coating may be replaced by a robust biocompatible alternative, such as biocompatible epoxies or stereolithography (SLA) 3D-printed shells comprised of surgical guide resin, which has been demonstrated in similar capsule platforms that traverse the GI environment.

### Example Experimentation of Hydrogen Sulfide Sensor

In one non-limiting example, an electrochemical H₂S sensor is comprised of three electrodes deposited on a flexible polyimide film (Kapton, 1 mil): a thin-film Au WE (4 mm diameter), Au CE, and an Ag RE. The concentric electrodes were designed using Autodesk AutoCAD and patterned via a series of paper shadow masks laser cut with a Glowforge Pro CO2 laser cutter. Prior to deposition, the mask was affixed to the polyimide substrate and baked in a furnace at 60° C. for 1 h to remove excess moisture. The exposed surface of the polyimide substrate was treated using an O₂ plasma cleaner with 4 SCCM of O₂ for 90 s at 150 W to improve thin-film adhesion. Metal layers of Cr/Au (20 nm/100 nm) were deposited using e-beam evaporation, followed by a separate deposition of Ag (300 nm) for the RE at deposition rates of 1.5, 2, and 2.5 Å s⁻¹, respectively. Sensors were cleaned with a combination of acetone, methanol, and isopropanol (AMI); rinsed thoroughly with DI water (>18.2 MΩ) from an E-pure Ultrapure Water Purification System, and dried with N₂. To confine the Nafion membrane, an acrylic (10 mil) reservoir backed with 9495MP double-sided tape was laser cut and attached above the sensor. The acrylic cutout (Ø=12 mm) had a circular opening (Ø=8 mm) positioned around the sensor electrodes, as well as three 1 mm ports aligned with the sensor contact pads. An identical acrylic layer (Ø=12 mm), without the 8 mm cutout, was affixed to the back of the substrate to support the sensor assembly. To interface the sensor with the capsule electronics, a 20-AWG needle was used to make small perforations through the 1 mm openings aligned with each contact pad. Au header pins were inserted through the openings and secured with Ag epoxy (8330S, Digikey), and cured at 45° C. for 24 h. All sensors were examined for shorted electrical connections between the contact pins, cleaned with AMI, rinsed in DI water, and finally dried with N₂. This assembly strategy enables modular integration of the miniaturized H₂S sensor within the ingestible capsule form factor.

For the formation of the solid-state polymer electrolyte, a 5% w/v Nafion resin (EW, 1100 g eq⁻¹) mixture of lower aliphatic alcohols and water was purchased from Sigma-Aldrich and used as received. The Nafion dispersion (20 μL) was mixed in its original container for 60 s and then drop-cast onto the surface of the Au sensor. The sensors and a paper filter soaked in 80% diluted ethanol were placed in a small plastic Petri dish (30 mL) and sealed. The Petri dish was kept at room temperature for 24 h to slowly evaporate solvents from the Nafion resin, and then placed in a furnace for 1 h at 80° C. to completely remove the remaining solvents. Controlling the rate of solvent evaporation in ensures a uniform distribution of Nafion across the electrodes, minimizing the risks of cracks forming in the film. The Nafion membranes were placed in a sealed humid container for 24 h to rehydrate. The films were functionalized by pretreatment with 0.1 m H₂SO₄ (50 μL) and resealed in the humid container for 48 h, allowing the acid to innervate the film. The sensor was then rinsed in DI water and stored in a humid chamber until use. Before use, a Teflon membrane (Ø=6 mm, pore size: 5 μm) was lightly pressed into contact with the Nafion film and sealed. The Teflon membrane functions as a gas-permeable, liquid-impermeable interface between the Nafion and the external environment, preventing sensor corrosion and fouling of the sensor.

A double-sided flex-rigid PCB was designed using Autodesk EAGLE and consists of two six-layer circular FR-4 ceramic substrates (Ø=12 mm) connected by a 15 mm long polyimide flex connecting region with embedded copper traces. Several COTS components were incorporated into the design: 1) an electrochemical AFE, AD5941, to excite the electrochemical sensor and record resulting current values using an onboard ADC and FRAM, 2) a BLE-MCU, BGM13S, and external 2.45 GHz ceramic chip antenna, WLA.01, for wireless data acquisition (signal power: 0-+18 dBm) and energy management, and 3) a 3.3 V voltage regulator, TPS610981, to maintain a constant operating voltage across all components. The system is powered by a 3.0 V, 160 mA h Li—MnO₂ coin-cell battery, 2L76, featuring a high capacity-to-size ratio. Battery connections were made using 30 AWG insulated copper wires soldered to the (+) and (−) terminal of the battery. A nickel cap was spot welded to the (+) side of the coin-cell to facilitate the solder joint and avoid damaging the battery. The 30 AWG wires were guided through a 3D-printed spacer and connected to the corresponding power pins via Au pin receptacle. Additionally, a 15 AT magnetic reed switch, HSR-502RT, is placed between the (+) battery terminal and the voltage regulator allowing the electronics to be turned on and off depending on the capsule's proximity to an external magnetic field, eliminating power consumption when in storage. Finally, three 22-AWG Au pins (5 mm) were mounted to the PCB to align and mount the WE, CE, and RE electrodes from the H₂S sensor. Only digital signals and power traces were routed between the two rigid substrates to minimize signal noise due to bending the embedded flex connector (bend radius: 1.2 mm)

The flex-rigid PCB, 3D-printed spacer, and battery were encapsulated in PDMS at a 10:1 monomer to curing agent ratio and baked at 65° C. for 24 h prior to removal. For PDMS encapsulation of the electronics, custom molds were designed in Fusion 360 and, using fused filament fabrication (FFF), were 3D-printed from PLA filament with a Prusa MK3S+3D-printer. The mold incorporated four embedded neodymium magnets to turn off the capsule during curing. The electronics were removed from the mold achieving a 14×34 mm² cylindrical capsule. Second, excess PDMS was removed to expose the Au pin connectors for the WE, CE, and RE electrodes, facilitating the connection of the H₂S sensor and molded capsule electronics. A PLA cap (Ø=14 mm) with a 6 mm opening was 3D printed to cover the Teflon protected sensor and was thermally melded to the rim of the cap to prevent liquid from shorting the sensor while still allowing gas to diffuse from the external environment. Finally, a PLA cap was sealed to the PDMS capsule with a biocompatible epoxy.

The custom-made gas testing set-up provides a humid environment for modulating various intraluminal gas species. To generate specific concentrations, nonflammable calibration tanks of H₂S (50 ppm, N₂-diluted), H₂ (3%, N₂-diluted), CO₂ (99.99%), and CH₄ (2.5%, air-diluted) respectively, were vented into a plastic test chamber (2400 mL) until the desired concentration was reached. Gas flow was regulated at two constant flow rates: 0.2 SLPM (standard liter per minute) for H₂S and 0.4 SLPM for all proposed interferent gases (H₂, CO₂, and CH₄). To return the chamber to an ambient baseline condition (air), an outlet valve was opened, and the chamber contents were removed using a vacuum pump. The required venting time to return the sensor to its baseline value was dependent on the gas concentration in the chamber, though purging for three 20 s pulses followed by a 15 s wait time was sufficient for all test values and preserved sensor lifetime. A Mega2560 Arduino development board and serial monitor was used to remotely control the test setup. A highly humid test environment was maintained using a water vapor atomizer (vapor rate: 380 mL h-1) for 1 min following each cycle of gas purging and venting, as high relative humidity is essential for a conductive Nafion membrane.

The concentration of gas in ppm was calculated according to Equation 1:

$$\frac{{C}_{Tank} [ \frac{mg}{L} ] \cdot Q [ \frac{L}{s} ] \cdot {t}_{i n} [ s ]}{{V}_{Cont}} = ppm$$

where V_{Cont} denotes the container volume, C_{Tank}, is the concentration of the gas calibration tank, Q is the flow rate of the calibration gas, and tin is the infill time required to achieve specific gas concentrations. The volume of the container (2400±30 mL) includes the volumetric sections of the test chamber and attached tubing. The initial ambient composition of the chamber is taken as air (79% N2 and 21% (2) at atmospheric pressure. N₂-diluted H₂S is introduced into the container at 0.2 SLPM, or 0.0033 L s-1, for 65 s. Interferent was added into the container at 0.4 SLPM, or 0.0067 L s-1, for 6 s. Data were presented as mean±standard deviation (SD). For gas sensing experiments, a sample size (N=3) corresponds to the measurement of unique sensors, while for wireless attenuation testing, the number of replications (N=60) refers to repeated measurements over time. Raw data collected from H₂S sensors and the capsule platform represents an output current response, including a 10 min “warmup time” to achieve a stable baseline current before the introduction of reactive gases. Data points recorded prior to the established “warm-up time” were excluded as outliers and not used in the evaluation of the sensor response. The remaining data points, after the “warm-up time,” were analyzed using MATLAB to determine the current response following a 3 min “wait-time” after a gas was introduced. Peak current values were normalized to the highest linear concentration achieved (4.5 ppm) for both calibration and selectivity evaluation. No statistical methods were used to assess significant differences.

1. A device for in vivo sensing of luminal concentration of a compound of interest within a gastrointestinal (GI) tract of a subject, the device comprising:
an ingestible housing with an outer profile to pass through the subject's GI tract;
a battery contained inside of the housing;
a communication module inside of the housing;
a sensor disposed on an outer surface of the housing, the sensor comprising one or more electrodes configured to contact luminal contents of the subject's GI tract while the sensor is in motion traversing through the subject's GI tract; and
a microcontroller in electrical communication with the one or more electrodes, wherein the microcontroller comprises a memory having software instructions stored thereon, which cause the microcontroller to:
control a plurality of electrical signals to the one or more electrodes, the plurality of electrical signals comprising attributes relating to a property of the compound of interest;
receive a continuous output signal from the sensor, indicative of an extent to which the compound of interest is present in the luminal contents and contacting the one or more electrodes as the sensor traverses the subject's GI tract; and
transmit time series information regarding presence of the compound of interest along the subject's GI tract via the communication module.

2. The device of claim 1, wherein the sensor is a Nafion-Au H₂S sensor.

3. The device of claim 1, wherein the sensor comprises a membrane covering the one or more electrodes.

4. The device of claim 3, wherein the membrane is liquid impermeable and gas permeable.

5. The device of claim 3, wherein the membrane is Teflon.

6. The device of claim 1, wherein the sensor is disposed on a leading end of the outer surface of the housing, relative to a direction of travel of the device through the subject's GI tract.

7. The device of claim 1, wherein the microcontroller causes the one or more electrodes to perform a voltage sweep.

8. The device of claim 1, wherein the microcontroller causes the one or more electrodes to apply a bias voltage.

9. The device of claim 1, wherein the sensor does not measure the presence of a compound within a lining of the subject's GI tract.

10. The device of claim 1, wherein the time series information sent by the microcontroller comprises a plurality of times stamps corresponding to the plurality of electrical signals from the sensor.

11. The device of claim 10, wherein the microcontroller generates timing information from which a position of the device in the subject's GI tract can be estimated based on the plurality of time stamps.

12. The device of claim 7, wherein the voltage sweep comprises a cyclic voltammetry between −0.1 V and 0.6 V and a scan speed of 100 mV/sec.

13. The device of claim 7, wherein the sensor detects a presence of Serotonin in a lumen of the subject's GI tract during the voltage sweep.

14. The device of claim 8, wherein the bias voltage is about-0.2 V.

15. The device of claim 8, wherein the sensor performs a measurement of one or more gases within the subject's GI tract when the bias voltage is applied.

16. The device of claim 15, wherein the measurement is an amperometric measurement of H₂S.

17. A method of manufacturing an in vivo sensor, the method comprising:
providing a capsule comprising an ingestible housing, wherein the ingestible housing is configured to continuously traverse through a subject's GI tract;
disposing a battery within the capsule;
disposing a communication link within the capsule;
forming one or more electrodes on an outer surface of the capsule, the one or more electrodes being configured to detect luminal concentration of a compound of interest within a gastrointestinal (GI) tract of a subject while the in vivo sensor continuously traverses the GI tract; and
integrating a microcontroller within the capsule, and electrically connecting the microcontroller with: the battery; the communication link; and the one or more electrodes.

18. The method of claim 17, further comprising applying a semipermeable membrane to the surface of the sensor.

19. The method of claim 18, wherein the semipermeable membrane is gas-permeable and liquid-impermeable.

20. The method of claim 17, further comprising providing software instructions stored on the microcontroller that cause the microcontroller to record values of an output signal of the one or more electrodes and associate at least a portion of the values with a time stamp.

21. The method of claim 20, further comprising providing software instructions stored on the microcontroller that cause the microcontroller to estimate a position of the capsule for a plurality of the values based on the time stamps.
